(19) United States
(12) Patent Application Publication
(10) Pub. No.: US 2004/0167544 A1
(43) Pub. Date: Aug. 26, 2004

(54) Progressive hair removal surface

(75) Inventor: Randy Nicolosi, Shelton, CT (US)

Correspondence Address: Richard R. Michaud, McCormick, Paulding & Huber LLP, CityPlace II, 185 Asylum Street, Hartford, CT, 06103, US

(73) Assignee: Eveready Battery Company, Inc., Westlake, OH

(21) Appl. No.: 10/733,997

(22) Filed: Dec. 11, 2003

Related U.S. Application Data
(60) Provisional application No. 60/433,709, filed on Dec. 13, 2002.

(51) Int. Cl. A61B 17/50

(52) U.S. Cl. 606/133

(57) ABSTRACT

In a progressive hair removal surface a substrate is provided which includes a plurality of protrusions defining surfaces collectively forming adjacent peaks and valleys. A plurality of abrasive members project from at least one of the surfaces forming the protrusion for progressively abrading hair from a hirsute surface as said hair removal surface is drawn there across.

### CROSS-REFERENCE TO RELATED APPLICATIONS

This application is entitled to the benefit of and incorporates by reference essential subject matter disclosed in Provisional Patent Application No. 60/433,709 filed on Dec. 13, 2002.

### FIELD OF THE INVENTION

The present invention is generally directed to the removal of hair from hirsute surfaces, and is more particularly related to an abrasive hair removal mechanism.

### BACKGROUND OF THE INVENTION

Historically, hair removal via shaving has been accomplished using extremely sharp blades generally referred to as razor blades. The blades are usually retained in a housing attached in some manner to a handle. During a shaving operation, the sharpened edge of the razor blade is drawn across a user's skin and cuts the hair as dose to the surface of the skin as possible. The closeness of a shave is largely dependent on how close to the skin the blade is able to cut hair protruding therefrom. To achieve a closer shave multiple passes can be made over the same area of skin making successive cuts of the hair, each reducing the amount by which the hair protrudes from the skin. Alternatively, multiple blades can be housed in the razor so that a hair is cut more than once in a single pass. However, the greater the number of passes over a particular area, the greater the potential for becoming cut by the razor blades as well as for discomfort resulting from the blades engaging the same area multiple times.

A drawback associated with the manufacture of conventional razors employing blades is that the blades, due to the extremely sharp cutting edges, create handling difficulties. Moreover, the mass production of these blades requires a great deal of equipment, and process control which translates into higher expenses and costs.

Furthermore, where hair is cut using a razor blade, the edge of the cut hair tends to be somewhat sharp so that although a close shave may have been achieved, when one runs a hand across the shaved surface, the surface can be tactilely rough. Conversely, if a frayed hair end is achieved, a softer more comfortable feel is accomplished.

Based on the foregoing, it is the general object of the present invention to provide a shaving surface that overcomes the problems and drawbacks of the prior art.

### SUMMARY OF THE INVENTION

The present invention is directed in one aspect to a progressive hair removal surface wherein a substrate includes a surface having a plurality of protrusions projecting therefrom. Each protrusion has surfaces that collectively define adjacent peaks and valleys. A plurality of abrasive members project from at least one of the surfaces and are adapted to progressively abrade hair projecting from a hirsute surface as said hair removal surface is drawn there across.

Preferably, the protrusions each define an approximately triangular cross-section formed by generally opposed inclined surfaces culminating in an apex or peak. In the preferred embodiment of the present invention, at least a portion of the apex of each protrusion is coated with a lubricious material such as, but not limited to, Polyox® to promote a smooth feel when the hair removal surface is drawn over a hirsute surface and to minimize discomfort to the user.

The substrate can be formed from a suitable material such as amorphous metal or polymer, however, the present invention is not limited in this regard as other materials such as stainless steel, tungsten, or a metal coated polymer can be substituted. In addition, the lubricious material can be in the form of a coating, or a piece of the material can be attached to the apex of one or more of the protrusions.

The protrusions can be elongated and arranged in at least one row with a base portion defined by each protrusion abutting the base portion of the next successive protrusion. The protrusions can also be pyramidal having four or more inclined surfaces, one or more of which have a plurality of abrasion members projecting outwardly therefrom. The protrusions can assume any one of a number of different shapes, such as cylindrical, or triangular so long as collectively they act to abrade hair.

### BRIEF DESCRIPTION OF THE DRAWINGS

FIG. 1 is an enlarged partial side elevational view of an embodiment of the progressive hair removal surface of the present invention.

FIG. 2 is a perspective view of the embodiment of FIG. 1.

FIG. 3 is an enlarged partial side elevational view of the embodiment of FIG. 1 showing the apex of a portion of the protrusions being formed by lubricious material.

FIG. 4 is a perspective view of another embodiment of the progressive hair removal surface of the present invention.

FIG. 5 is an enlarged view of the embodiment shown in FIG. 4.

### DETAILED DESCRIPTION OF THE PREFERRED EMBODIMENTS

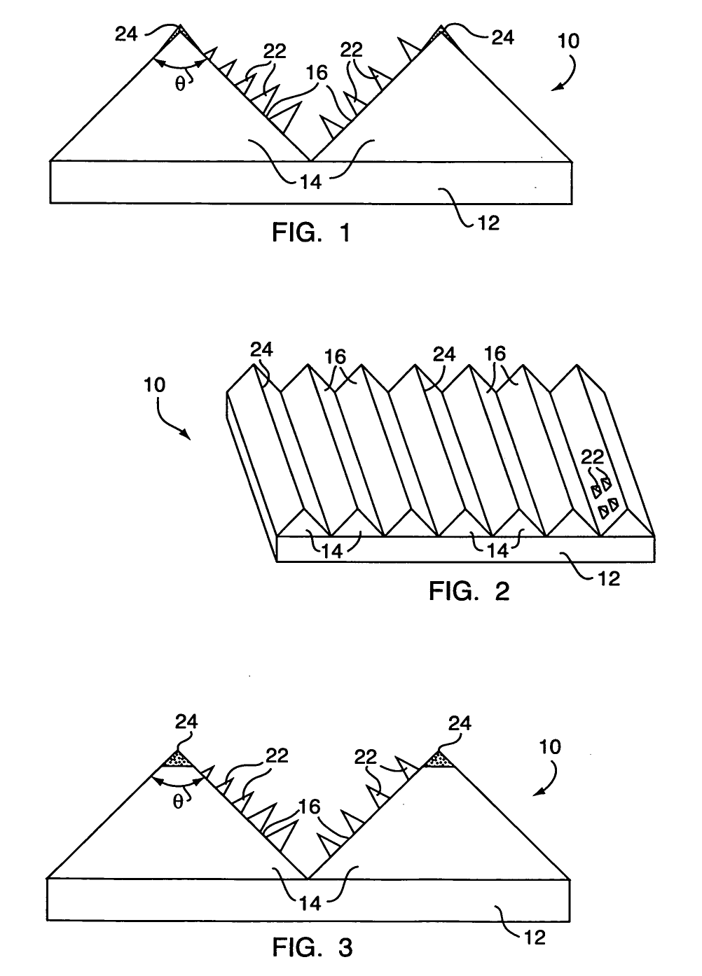

As shown in FIG. 1, a progressive hair removal surface is generally designated by the reference number 10 and includes a substrate 12 from which a plurality of protrusions 14 outwardly extend, each protrusion defines surfaces 16 which collectively form a plurality of adjacent peaks and valleys. A number of abrasive members 22 project outwardly from at least some of these surfaces 16 for progressively abrading hair from a hirsute surface as the progressive hair removal surface is drawn there across during a shaving operation. While the abrasive members 14 are shown in the illustrated embodiment being of different sizes, the present invention is not limited in this regard as any number of abrasive members randomly or uniformly sized can be employed without departing from the broader aspects of the present invention.

In the illustrated embodiment, the protrusions 14 are shown as being isosceles triangles with the angle θ being the same for all intersecting sides of each triangular surface, the present invention is not so limited. The members do not have to be triangular in shape nor is it requisite that they have sides or surfaces 16 being of equal length and uniformly angularly positioned. Different geometric shapes and angular configurations can be employed without departing from the present invention. In the embodiment of the present invention shown in FIG. 2 each protrusion 14 is illustrated as being elongated and triangular in cross-section. In addition, the protrusions 14 are arranged in a row with each protrusion being approximately parallel to the next successive protrusion 14.

Referring now to both FIGS. 1 and 2, each of the protrusions defines a peak portion 24 which in the illustrated embodiment is coated with a lubricious material such as, but not limited to, Polyox® to allow the progressive hair removal surface to be drawn comfortably across the skin of a user during the shaving operation. While the illustrated embodiment shows each of the peak portions or apexes 24 as being coated with a lubricious material, the present invention is not limited in this regard as only a portion of the apexes may be coated with the abrasive material. In addition, as shown in FIG. 3, the entire apex can be formed from lubricious material. Still referring to FIG. 2, the abrasive members protruding from the surface 16 are shown as being pyramid shaped. However, the invention is not limited in this regard as these abrasive members can be conically shaped or can extend along the entire length of the protrusion and be defined by a triangular or other cross-section without departing from the broader aspects of the present invention.

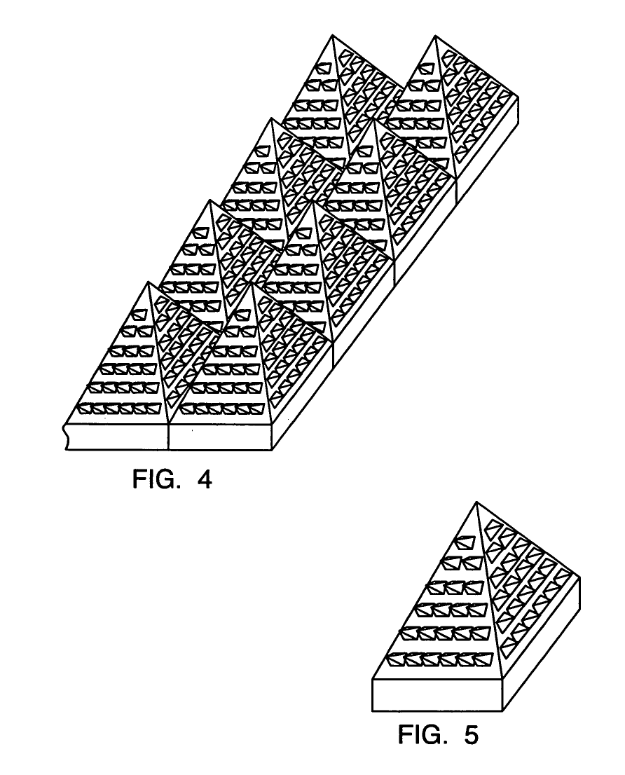

Turning next to FIGS. 4 and 5, the progressive hair removal surface of the present invention can also be prismatic in composition with a plurality of individual protrusions abutting one another along a base portion of each and having a plurality of abrasive members extending from one or more of the surfaces comprising each prismatic member.

During operation, the progressive hair removal surface of the present invention can be mounted into any of a number of different shaving heads to allow the surface to be positioned so as to be easily drawn across a hirsute surface. As the progressive hair removal surface engages the hirsute surface, the abrasive members each abraid any hair in which they come in contact thereby allowing a user to achieve a close shave with the added advantage that the abrasive nature of the progressive hair removal surface leaves a frayed hair end which in turn yields a softer and smoother feel to the shaved skin.

While preferred embodiments have been shown and described, one skilled in the pertinent art to which the present invention pertains will immediately recognize the various modifications and substitutions may be made. Accordingly, it is to be understood that the present invention has been described by way of example, and not by limitation.

What is claimed is:

1. A progressive hair removal surface comprising:
a substrate, a surface of which includes a plurality of protrusions defining surfaces collectively forming adjacent peaks and valleys;
a plurality of abrasive members projecting from at least one of said surfaces for progressively abrading hair from a hirsute surface as said hair removal surface is drawn there across.

2. A progressive hair removal surface as defined by claim 1, wherein:
said protrusions each define an approximately triangular cross-section having an apex; and wherein
at least a portion of said apexes are coated with a lubricious material.

3. A progressive hair removal surface as defined by claim 1, wherein:
said protrusions each define an approximately triangular cross-section having a tip portion formed from a lubricious material.

4. A progressive hair removal surface as defined by claim 1, wherein said substrate is metallic.

5. A progressive hair removal surface as defined by claim 4, wherein said substrate is formed from amorphous metal.

6. A progressive hair removal surface as defined by claim 1, wherein said substrate is polymeric.

7. A progressive hair removal surface as defined by claim 1, wherein:
each of said protrusions is pyramidal and defined by at least four inclined surfaces; and wherein
said abrasive members project outwardly from each of said inclined surfaces.

8. A progressive hair removal surface as defined by claim 1, wherein;
said protrusions are arranged in at least one row, with each protrusion being elongated and defining an approximately triangular cross section having at least two generally opposed inclined surfaces; and wherein
each of said inclined surface includes a plurality of said abrasive members projecting outwardly therefrom.

9. A progressive hair removal surface as defined by claim 7, wherein at least a portion of said protrusions define a lubricious tip portion.

10. A progressive hair removal surface as defined by claim 8, wherein at least a portion of said protrusions define a lubricious tip portion.

11. A progressive hair removal surface as defined by caim 1, wherein each of said protrusions defines a sharpened edge.

12. A progressive hair removal surface comprising:
a substrate having a plurality of protrusions defined by inclined surfaces defining adjacent peaks and valleys;
a plurality of abrasive members projecting outwardly from said inclined surfaces each adapted to abrade hair from a hirsute surface; and
a portion of said peaks being formed at least in part by a lubricious material.
